(19) United States
(12) Patent Application Publication
(10) Pub. No.: US 2021/0137547 A1
(43) Pub. Date: May 13, 2021

(54) STEERABLE ENDOLUMINAL PUNCH WITH CUTTING STYLET

(71) Applicant: Indian Wells Medical, Inc., Lake Forest, CA (US)

(72) Inventors: James A. Carroll, Long Beach, CA (US); Jay A. Lenker, Lake Forest, CA (US)

(73) Assignee: Indian Wells Medical, Inc., Lake Forest, CA (US)

(21) Appl. No.: 17/152,611

(22) Filed: Jan. 19, 2021

Related U.S. Application Data
(63) Continuation of application No. 16/132,027, filed on Sep. 14, 2018, now abandoned.
(60) Provisional application No. 62/558,768, filed on Sep. 14, 2017.

(51) Int. Cl. A61B 17/32; A61B 17/34; A61B 90/00

(52) U.S. Cl. CPC A61B 17/32002; A61B 17/3478; A61B 2017/00247; A61B 90/39; A61B 17/3476; A61B 17/320068

(57) ABSTRACT

An endoluminal punch with a penetrating stylet and a vibration generator. The vibration generator is operable to cause vibrations and rapid reciprocation of the distal tip of the stylet to facilitate penetration of the stylet through resistant body tissue such as the fossa ovalis. The vibration generator may also be operable to cause vibration of the distal tip of the punch itself, to facilitate passage of the punch through an initial perforation created by the stylet.

This application is a continuation of U.S. application Ser. No. 16/132,027, filed Sep. 14, 2018, now pending, which claims priority to U.S. provisional application 62/558,786, filed 14 Sep. 2017, expired, the entirety of which are hereby incorporated herein by reference.

### FIELD OF THE INVENTIONS

The inventions described below relate to devices and methods for endoluminal punches for penetrating the fossa ovalis of a patient.

### BACKGROUND

The currently accepted procedure for gaining access to the left atrium, in a minimally invasive procedure, involves routing a needle called a Brockenbrough needle into the right atrium, through the fossa ovalis. The Brockenbrough needle is pre-placed within a guiding catheter or dilator. The guiding catheter specifically preferred for use with a Brockenbrough needle is called a Mullins catheter or transseptal introducer. The Brockenbrough needle is a long, small diameter punch, generally formed from a stainless steel wire stylet that is surrounded by a stainless steel tube. The stylet is used to make an initial incision in the fossa ovalis, and the punch is then pushed through this initial incision, and a dilator is then pushed over the punch to provide access to the left atrium. The fossa ovalis is sometime quite tough, and resistant to penetration by the stylet. Even after penetration by the style, the tissue of the fossa ovalis around the stylet can be pulled taut and maintain high strength such that it may catch on the beveled tip of a needle. This may hinder or even prevent needle penetration of the pierced tissue.

### SUMMARY

The devices and methods described below provide for easier penetration of the fossa ovalis with an endoluminal punch. The devices include an endoluminal punch with a stylet disposed within a lumen of the punch, and mechanisms for vibrating or longitudinally reciprocating the distal tip of the stylet while it is pressed against the fossa ovalis, to reduce the force necessary to penetrate the fossa ovalis. The endoluminal punch may also include a mechanism for rotating the stylet within the lumen of the punch, to cause radially oriented vibration of the distal tip of a tube of the punch, again while holding the punch against the fossa ovalis, to further reduce the force necessary to penetrate the fossa ovalis, and overcome resistance to passage of the tube of the punch. The mechanism for reciprocating the stylet of the endoluminal punch may be configured to allow removal of the stylet after the endoluminal punch has been passed through the fossa ovalis. By vibrating the distal tip of the stylet, the force necessary to penetrate the fossa ovalis is greatly reduced. By vibrating the distal tip of the tube of the punch, the force necessary to overcome any snagging or catching of the punch while being forced through the fossa ovalis is greatly reduced.

### BRIEF DESCRIPTION OF THE DRAWINGS

FIGS. 1A and 1B illustrates side views of an endoluminal punch, in partial breakaway view, having a tubular needle, a handle, and a vibration generator.

FIG. 2 illustrates the endoluminal punch of FIGS. 1A and 1B within an introducer sheath and dilator.

FIGS. 3A and 3B illustrates a steerable endoluminal punch with a vibration generator attached to the handle configured to vibrate the endoluminal punch itself.

FIGS. 4A and 4B illustrates a spade bit tip for the piercing stylet.

FIGS. 5A and 5B illustrates curved tips for the piercing stylet.

FIGS. 6A and 6B illustrates a screw tip for the piercing stylet.

FIG. 7 illustrates the hub of a steerable transseptal needle further comprising a piercing stylet hub having a pushbutton that also generates rotary motion as the stylet shaft is being advanced and retracted axially.

FIG. 8 illustrates the distal tip of a piercing stylet comprising a hollow tube a slot, and a blade affixed within the slot of the hollow tube but able to rotate radially outwardly.

FIG. 9 illustrates a piercing stylet distal tip comprising a pointed end and a plurality of circumferential grooves proximally disposed to the pointed end, according to an embodiment of the invention.

### DETAILED DESCRIPTION

FIG. 1A illustrates a side view of an endoluminal punch comprising an outer tube 102, a layer of optional sound insulation 122, an optional tubular inner shaft 116, a distal tip 104, a hub or handle 106, a vibration generator 108. The punch assembly 100 also comprises a stylet or obturator wire 114 further comprising a handle or hub 118, and a distal tip 120. The outer tube 102 is affixed to the hub or handle 106. The vibration generator 108 is affixed to the hub 108 or it can be affixed to the outer tube 102, optionally the tubular inner shaft 116, or both. The stylet 114 is preferably slidably disposed within a lumen 128 of inner tube 116, and may be removable entirely from the inner tube, or may be secured (though slidable) within the inner tube. The vibration generator is longitudinally fixed (and may also be rotationally fixed) to the inner tube, the outer tube, or both, and coupled to the stylet so that it may rapidly vibrate, or longitudinally reciprocate, the stylet relative to the inner tube and outer tube. The vibration generator may be manually operated, or operated by one of the several powered devices described below. For embodiments using a powered device as a vibration generator, the controller 110 may be operably connected to the vibration generator 108, as well as to any necessary power supply 112 by the electrical bus 126.

An example of a suitable vibration generator is illustrated in FIGS. 1A and 1B. This vibration generator comprises a motor 130, coupled to a rotary cam 131, which is in turn coupled to a rotary cam follower 132, which is longitudinally fixed to the stylet 114 at its proximal end (through collar 136), and preferably rotationally fixed, or selectively fixable, within the vibration generator. The motor may be operated to rotate the cam, which in turn forces the cam follower distally, repeatedly as the cam rotates, to cause the desired rapid reciprocating movement of the stylet distal tip. The cam follower may be biased (spring biased) to return proximally, or may be positively forced proximally by the cam. If the stylet is to be non-removable from the remainder of the punch, the stylet proximal end may be non-removably fixed to the cam follower. If the stylet is to be removable from the reminder of the punch (to allow for use of the inner tube lumen for other devices, or for fluid delivery), the motor can comprise a hollow shaft motor, with hollow shaft 133, and the stylet can be longitudinally fixed to the cam follower with means, such as a set screw or toggle bolt 134 accessible from the outside of the vibration generator, for selectively longitudinally fixed the stylet to the cam follower for operation, and selectively releasing the stylet from the cam follower so that it may be removed. Thus, the system may be assembled with the stylet longitudinally fixed to the cam follower, and longitudinally translatable relative to the inner tube and outer tube of the punch. Other cam arrangements, such as a positive drive cam with a motor shaft perpendicular to the long axis of the punch, or a preloaded spring cam, or a cylindrical cam, may be employed.

The endoluminal punch may be steerable (with a distal deflectable segment selectively bendable by an operator, through a mechanism in the proximal end of the punch) or non-steerable (so that it would be deflectable only due to forces applied by the anatomy or a surrounding introducer or guide catheter). In steerable embodiments, the punch may be constructed as shown in our prior U.S Patent, Lenker, Steerable Endoluminal Punch, U.S. Pat. No. 8,961,550 (Feb. 24, 2015) (the entirety of which is hereby incorporated by reference), with the inner tube longitudinally fixed, at its distal end, to the outer tube at its distal end, with mechanisms in the proximal hub for tensioning the inner tube relative to the outer tube to effect steering. The advantages of the reciprocating stylet, then, can be achieved with or without the advantages of steering mechanisms. Also, if the endoluminal punch is not configured for steerability, then one of the inner or outer tubes of the punch may be omitted, and the stylet may be disposed within a single tube comprising the punch (which, in turn, might be disposed with a dilator and/or introducer, as shown in FIG. 2).

FIG. 1B illustrates a side view of the punch, needle, or catheter assembly 100, with the stylet or obturator wire (114 and 118) removed. The endoluminal punch 100 comprises the inner tube 116, the layer of optional sound insulation 122, the optional outer tube 102, the distal tip 104, the hub or handle 106, the vibration generator 108, the controller 110, the electrical bus 126, and the power supply 112. The punch assembly 100 also comprises a switch 124. The outer tube 102 is affixed to the hub or handle 106.

In lieu of the cam operated embodiment illustrated in FIGS. 1A and 1B, the vibration generator 108 can comprise a voice coil actuator or voice coil motor, an ultrasonic transducer, a sonic transducer, a linear vibration generator, a motor with spinning out of balance weights, or the like, couple to the stylet proximal end. Each of these may be non-removably attached to the stylet proximal end or removably attached to the stylet and longitudinally fixed, or released, from the stylet using set screws, toggle bolts, bayonet fittings or comparable means. The amount of longitudinal motion to be applied (total travel) can range from about 0.005 inches to about 0.25 inches and preferably between about 0.005 and 0.1 inches. Rate of vibration or reciprocating motion is preferably in the range of 1 Hertz to 20 kilohertz, and may be variable through control of the motor speed (relative to FIG. 1A, for example) or variation in the signal supplied to a voice coil, for example. The power level of the vibration device can be in the range of about 1 watt to about 100 watts with a preferred range of about 5 watts to about 50 watts and a more preferred range of about 5 watts to about 20 watts.

FIG. 1A illustrates additional features which provide for vibration of the distal tip 114 of the inner tube 116, which can further facilitate penetration of the punch through the fossa ovalis. These features include an eccentric radial protrusion 135 near the distal tip of the stylet, longitudinally aligned with the bevel of the sharp distal tip 114 of the inner tube 116. The protrusion may be fixed (especially if the stylet is not removable) or retractable (from of an elastically biased protruding wire, or an outwardly biased leaf for example). The protrusion may be formed by a slight bend in the stylet or slight curvature in the stylet proximate the bevel (a slight bend or curvature, if resilient, will allow for insertion and removal of the stylet), such that the stylet has a uniform diameter proximate the distal end of the first tube, and the eccentric radial protrusion comprises a curvature in the stylet, and the stylet is resiliently deformable such that the distal tip of the stylet, including the eccentric radial protrusion, may be drawn proximally through the first tube. Several variations in the distal end of the stylet may also be employed to create a wobbling eccentric motion of the distal end of the stylet to impart vibration to the distal tip of the inner tube. For example, the stylet may comprise a region of denser material to provide a non-uniform weight distribution through the cross section of the stylet (not protruding beyond the diameter of the stylet or inner tube lumen), or the stylet may comprise a half-cylinder in the region of the beveled distal tip of the inner tube, to create an imbalance during rotation.

The protrusion, bend or curvature is located along the length of the stylet, relative to the bevel, such that, when the stylet is disposed with the distal tip 120 distal to the bevel, or longitudinally aligned with the bevel, and is rotated, the protrusion repeatedly impacts the long end of the bevel, to cause a wobbling radial vibration of the distal tip of the inner tube. (This wobbling vibration may be sufficient, in conjunction with the sharp beveled tip of the inner tube, that it may employed without also employing the stylet to create an initial perforation in the target body tissue, so that the distal tip of the stylet need not extend beyond the distal tip of the inner tube, so long as the eccentric structure is longitudinally aligned to cause vibration of the distal tip of the inner tube.)

To provide for rotating, non-reciprocating motion of the stylet while still maintaining the ability to provide reciprocating, non-rotating motion of the stylet, the cam may be shifted proximally (out of contact with the cam follower) and fixed to the stylet or follower temporarily, through a set screw or toggle bolt 137 or other mechanism or means for temporarily locking the cam to the stylet or to the follower (indirectly, as shown, or directly). For this functionality, the cam follower may be selectively fixed rotationally to the vibration generator, and selective allowed to rotate, and then both the cam and cam follower can be longitudinally fixed to the stylet, so that rotation of the cam results in rotation of the stylet and the cam follower (without resulting in reciprocation). To provide for rotating, non-reciprocating motion of the stylet without the ability to provide reciprocating, non-rotating motion of the stylet, the stylet can be secured to the motor directly and the cam and cam follower may be omitted.

To provide for rotating, non-reciprocating motion of the stylet while retaining the ability to provide reciprocating, non-rotating motion of the stylet, the stylet can be rotationally secured to both the cam (using set screw 137) and shifting the cam away from impinging relationship with the cam follower.

To provide reciprocating motion of the stylet, without the ability to remove the stylet, the stylet may be permanently fixed longitudinally (and, optionally, rotationally fixed) to the cam follower 132 (and item 118 may be omitted)(in such an embodiment, the stylet could be rotated by fixing it rotationally to the cam follower (directly, or indirectly), by fixing the motor rotationally to the stylet with set screw 138, or fixing the cam to the stylet using set screw 137). Thus, the cam, cam follower, motor shaft and stylet can be fixed or unfixed longitudinally and/or radially, at the selection of the manufacturer of the device, or the user of the device, to achieve various modes of operation.

Other arrangements can provide for reciprocating motion, such as a voice coil, solenoid or transducer, and, if rotation is desired, a motor may be coupled to the voice coil or transducer so that may rotate the voice coil or transducer and stylet together, or may be coupled to the stylet alone, to rotate the stylet while leaving the voice coil or transducer rotationally fixed. For example, the cam and cam follower arrangement of FIG. 1 can be replaced with a voice coil or solenoid, with the non-moving components of the voice coil longitudinally fixed to the inner and/or outer tube, and moving/translating components fixed to the stylet, so that operation of the voice coil/solenoid will result in reciprocating movement of the stylet within the punch. (The moving component may be either the magnet or the coil of the voice coil). The moving component may, like the cam assembly, include a pass-through for the stylet, so that it may be removable from the punch, and so that a motor may be employed to rotate the stylet.

FIG. 2 illustrates a vibratory endoluminal punch 100 inserted within a central lumen of an obturator or dilator 216 comprised by a guide catheter or introducer 200. The guide catheter or introducer 200 comprises an introducer tube 202, further comprising a central through lumen 216, tapered distal end 206, a hub 210, an obturator or dilator 204 further comprising a tapered distal end 208, a hub 212, and a central through lumen 214. The introducer hub 210 is affixed to the proximal end of the introducer tube 202. The introducer hub 210 can comprise hemostasis valve within the central lumen and a side port 218 which can be terminated with connectors such as Luer lock connectors, stopcocks, caps, and the like. The dilator hub 212 can comprise a hemostasis valve, sideports, and the like and can be terminated at its proximal end with a male Luer lock or other mating connector. The endoluminal punch 100 is slidably disposed within the central lumen 216 of the dilator, so that it may be advanced, withdrawn or removed from the dilator at the discretion of the user. Though the diameter of the punch 100 is smaller than that of the outside diameter of the introducer tube 202 or dilator 204, an initial perforation created by the punch is sufficient to ease penetration of the larger diameter dilator or introducer through the fossa ovalis or other tissue wall. The guide catheter, introducer, dilator, or other sheathing system can be used to isolate this motion, in whole or in part, from the patient and the user, generally along its entire length except at the distal tip. Thus, while the introducer 200 is held by the user and is maintained stationary with respect to the patient, the punch 100 can be moved axially therewithin to generate the incision mechanics caused by the sharp distal tip 104 and a gentle pounding motion directed generally perpendicular to tissue being penetrated.

Thus, as described in relation to FIGS. 1A, 1B and 2, the endoluminal punch comprises a first tube having a proximal end, and distal end, and a lumen extending from said proximal end to said distal end, with a stylet disposed within the lumen of the first tube, with a distal tip extending distally beyond the distal end of the first tube, and a vibration generator longitudinally fixed to the first tube, and coupled to the stylet, which is operable cause reciprocating motion of the stylet within the lumen of the first tube. The vibration generator can comprise the cam assembly illustrated in FIGS. 1A and 1B, operable to translate the stylet distally and proximally relative to the first tube. The vibration generator can comprise a voice coil, solenoid, transducer or linear actuator, eccentric weight driven by a motor, or other means, operable to translate the stylet distally and proximally relative to the first tube. The punch can comprise a second tube, disposed about the first tube, with means for tensioning one tube relative to the other, so that the distal end of the punch can be steered by tensioning one tube relative to the other.

The endoluminal punch can also comprise, in addition to or in lieu of the components of the vibrating stylet embodiment, components for vibrating the distal tip of the first tube, including the first tube with a beveled distal tip (or otherwise eccentric tip), a stylet with an eccentric radial protrusion or an eccentric distal tip, proximate the distal tip of the first tube, such that the stylet may be rotated to cause vibration of the distal tip of the first tube. This embodiment may include a motor for rotating the stylet, and may be coupled with the vibration generator longitudinally fixed to the first tube, and coupled to the stylet, to cause reciprocating motion of the stylet within the lumen of the first tube. The eccentricity of the stylet may be achieved with a stylet that has a uniform diameter proximate the distal end of the first tube, with the eccentric radial protrusion may comprise a curvature in the stylet, and the stylet may be resiliently deformable such that the distal tip of the stylet, including the eccentric radial protrusion, may be drawn proximally through the first tube. The eccentric radial protrusion may instead comprise region of non-uniform diameter, with a bulge that may have a slightly larger diameter that the inner diameter of the first tube.

FIGS. 3A and 3B illustrate side views of an endoluminal punch 300, in a steerable embodiment. FIG. 3A shows this steerable punch 300 in its initial, straight configuration, comprising a hub 302, a main tube 304, a first bendable region 306, a pressure shroud 308, a second bendable region 310, an inner tube 312 extending distally to the main tube 304, a distal end 314, the vibration generator 108, the stylet handle 118, the stylet 114, a stylet tip 120, the generator controller 110, the power supply 112, the electrical bus 126 and an actuation switch 124 (not shown).

Referring to FIG. 3A, the vibration generator 108 is affixed to the hub 302 of the steerable needle. The vibration generator shakes the hub 302 and its attached outer tube 304. The distal tip 314 of the stylet is sharp and capable of cutting, which can be accentuated by use of the vibrational driver 108. Rotation of the control knob 322 causes the bendable region 306 to articulate out of the plane under user control and guidance from fluoroscopy, echo, MRI, CT scan, real time ultrasound imaging, or the like.

FIG. 3B illustrates a side view of the steerable vibratory endoluminal punch 300 with its distal end having been articulated or bent out of the axial plane. The punch 300 comprises the hub 302, the main tube 304, the first bendable region 306, an optional pressure shroud 308, than optional second bendable region 310, the inner tube 312 extending distally to the outer tube 304, a distal end 320, a control knob 322, the vibration generator 108, a piercing stylet handle 334, a piercing stylet wire 330, a piercing stylet tip 332, a piercing stylet coupler 336, the generator controller 110, the power supply 112, the electrical bus 126 and the actuation switch 124 (not shown).

Referring to FIG. 3B, the vibration generator 108 is affixed to the handle 334 of the piercing stylet shaft 330. The handle 334 can be secured relative to the needle hub 302 by a coupler 336 which allows for vibratory movement but otherwise maintains a controlled spacing between the elements. In some embodiments, the vibration generator 108 can move the piercing stylet handle 334, the piercing shaft 330, and the piercing stylet tip 332 relative to the needle hub 302 such that the piercing stylet tip 332 can be cyclically driven distally toward tissue and proximally away from tissue. Articulation of the distal end is accomplished by turning the control knob 322 and bending occurs at the first bendable region 306, the second bendable region 310, or both. The pressure shroud 308 may be used to maintain fluid path integrity (should it be desirable to use a lumen of the punch to deliver contrast agent or other fluid) even in the presence of perforations in the inner tube or outer tube near the distal end.

In operation, the endoluminal punch may be operated similarly to the standard endoluminal punch, augmented with the step of operating the vibration generator to cause the stylet to longitudinally vibrate, reciprocate, while pressed against the fossa ovalis. The procedure is to advance an endoluminal punch, with a tissue piercing stylet affixed in place, through a transseptal introducer that has already been placed. The steerable transseptal needle can be straight, pre-curved, or it can be articulated to generate the proper curve, as determined under fluoroscopic or ultrasound guidance. The endoluminal punch introducer assembly is targeted within the right atrium of the heart at the fossa ovalis. Proper location, orientation, tenting, and other features are confirmed. Radiopaque dye can be injected through the steerable transseptal needle to facilitate marking of the fossa ovalis or blood flow around the distal end of the steerable transseptal needle. Pressure measurements can also be taken through the lumen of the steerable transseptal needle to confirm tracings consistent with the right or left atrium of the heart. Once proper positioning has been confirmed the stylet can be removed from the punch. Following removal of the stylet, the distal end of the punch can incise tissue and allow penetration through organ or luminal structures. However, the transseptal introducer is generally much larger in diameter than the endoluminal punch and may not be able to be forced through tissue that is particular thick, scarred, or highly elastic. At this point, the vibration generator can be engaged to shake or vibrate the endoluminal punch, including its distal tip. This shaking or vibration can increase the size of the tissue incision being created by the endoluminal punch such that it is easier to pass the introducer and dilator therethrough. Structures can be created on the exterior walls of the endoluminal punch, proximate its distal end, to facilitate tissue cutting when the distal tip is being vibrated. The method may be modified as appropriate, to punch through other tissue in the body.

As mentioned in relation to FIGS. 1A and 1B, a motorized or manually turned knob that is rotationally fixed to the stylet shaft can be advantageously used to pierce tissue with a corkscrew or threaded piercing stylet tip. The motor can provide for continual rotational motion, intermittent rotary motion, or oscillatory rotary motion. The oscillatory rotary motion can range from about 45 degrees to about 1,800 degrees per cycle (5 rotations) with a preferred range of about 90 degrees to about 720 degrees (2 rotations). The motor or the manual knob turn can be referenced to the hub of the transseptal needle. Various modification of the stylet tip may be employed, and these are described in the following description of FIGS. 4A through 9.

FIG. 4A illustrates a side view, in partial cutaway, of the distal tip of a steerable transseptal needle 400 comprising an outer tube 102, an inner tube 116 further comprising a lumen 412, a distal conic 410, a distal point 408, a punch shaft 402, a punch wire spade bit 404 and a sharp punch end 406. The punch shaft is slidably disposed within the inner tube lumen 412 and controlled by a mechanism at the proximal end of the steerable transseptal needle 400.

The spade bit 404 and sharp tip 406 can be affixed to the punch shaft 402 by welding, bonding, mechanical fasteners, or the like or they can be integrally formed. Shaping of the spade bit 404 can be accomplished by methods such as, but not limited to, laser cutting, grinding, EDM, and the like.

FIG. 4B illustrates an axial view looking at the distal end of the punch which comprises the shaft 402, the spade bit 404, and the sharp tip 406. In the illustrated embodiment, the sharp tip 406 is a 4 sided trocar but it can also be a conical shape or other more complex sharp structure.

In practice, the punch shaft 402 can be rotated by hand, by a motor, or the like. The punch shaft can rotate continuously, for a short duration, in a circumferential reciprocating fashion, or the like. One or more of the edges of the spade bit 404 can be sharpened to incise, cut, or otherwise damage tissue as the spade bit 404 is rotated, thus allowing for passage of larger structures such as the transseptal needle, an introducer, a dilator, and the like.

FIG. 5A illustrates one embodiment of the stylet distal end mentioned in relation to FIGS. 1A and 1B. The stylet 114 is disposed within the inner tube 116, which in turn is disposed within the outer tube 102. The eccentric radial protrusion 135 in this embodiment comprises a curvature in the stylet, and the stylet is resiliently deformable such that the distal tip of the stylet, including the eccentric radial protrusion, may be drawn proximally through the first tube. The protrusion is disposed near the distal tip of the stylet, longitudinally aligned with the bevel of the sharp distal tip 114 of the inner tube 116. The distal segment of the stylet is preferably resilient, so that it may be inserted through, and withdrawn through, the inner tube 116. This stylet has a uniform diameter proximate the distal end of the first tube.

FIG. 5B illustrates a side view, in partial cutaway, of the distal tip of a steerable transseptal needle 100 comprising an outer tube 102, an inner tube 116 with a beveled end 104, and a stylet 114 with spiral wire section 504 and a sharp tip 506. The stylet 114 is slidably disposed within the inner tube 116 and controlled by a mechanism at the proximal end of the steerable transseptal needle 100 to rotate circumferentially. The spiral wire end 504, similar to a corkscrew, is capable of penetrating tissue and then pulling the tissue toward the inner tube beveled tip 104 as it rotates, with reduced or no tenting of the myocardial tissue. The spiral wire can be rotated manually or motorized action at the proximal end hub or it can be moved axially in an oscillatory fashion, or both.

FIG. 6A illustrates a side view, in partial cutaway, of the distal tip of a steerable transseptal needle 600 comprising an outer tube 102, an inner tube 116 further comprising a lumen 412 and a distal point 408, a punch shaft 602, a threaded punch end 604 further comprising threads 608 and a sharp punch tip 606. The punch shaft 602 is slidably disposed within the inner tube lumen 412 and controlled by a mechanism at the proximal end of the steerable transseptal needle 600 to rotate circumferentially.

The punch shaft 602 can be advanced and retracted linearly so that the outside of the threaded punch end 604 grabs and cuts tissue against the distal end point 408. In other embodiments, the punch shaft 602 can be rotated to drive the threaded end 604 into tissue. This action can assist with pulling the tissue proximally toward the tip 408 and increasing the opportunity to advance the more proximal portions of the system 600, as well as a transseptal introducer, across and through the tissue being penetrated. In yet other embodiments, the punch shaft 602 can be both rotated about the longitudinal axis and oscillated axially. The punch tip 606 can be retracted within the inner tube lumen 412 so that it does not project beyond the tip 408 until such time as the operator is ready to begin the tissue penetration procedure. The threaded region 604 comprises threads 608 that can be either formed as left hand threads or right hand threads. The walls of the threads 608 are tapered to form a V-shaped valley between the threads. The sharp punch tip can be formed as a spade bit, a trocar, a conical section, or the like.

FIG. 6B illustrates a side view, in partial cutaway, of the distal tip of a steerable transseptal needle 600 comprising an outer tube 102, an inner tube 116 further comprising a lumen 412 and a distal point 408, a punch shaft 602, a threaded punch end 604 further comprising threads 610 and a sharp punch tip 606. The threads 610 are cut with steep walls, straight, vertical walls, or even undercut. This will permit a sharper edge in the thread area 604 and improved ability to cut tissue relative to the tapered threads shown in FIG. 6A.

FIG. 7 illustrates the hub end of a steerable transseptal needle 700 comprising a piercing stylet button 702, a piercing stylet rotator coupling 704, a rotational bearing or bushing 706, a wire torquer 720, a male Luer lock attachment 718, a main hub body 710, a control knob 712, an outer tube 714, an inner tube and control rods 716, a piercing stylet or punch shaft 708, a directional pointer 722, a rotation indicator 724, a Luer lock capped sideport 726, a stopcock 728, and a deflection gauge 730.

The piercing stylet button 702 is longitudinally affixed to the wire torquer 720 but can rotate freely relative to the wire torquer 720 due to the rotational bearing or bushing 706. Axial pressure on the button 702 causes the wire torquer 720 to advance longitudinally but also spin about its axis due to threads engaged on the stylet rotator coupling 704. The wire torque 720 is longitudinally and rotationally affixed to the piercing stylet shaft 708 so that the two components move together. A spring, not shown, can be used to return the piercing stylet wire torque 720 to its initial position after pressure is released from the button 702. In other embodiments, where rotation is not desired or required, the button 702 can be affixed to the wire torquer 720 without the rotational coupling bearing or bushing 706. The piercing stylet assembly can be removably affixed to the Luer lock proximal end of the stopcock 728 by way of the male Luer lock attachment or coupling 718 or other type of coupling.

The directional pointer 722 can be aligned with the direction of curvature of the distal end of the transseptal needle. The rotation indicator 724 can be used to show the user which direction to turn the control knob to cause deflection at the distal end of the transseptal needle. The deflection gauge 730 can be coupled to internal components, such as a jackscrew part (not shown) such that the deflection gauge 730 can represent the amount of internal movement and thus be related to unrestrained curvature angle at the distal tip of the steerable transseptal needle. The deflection gauge can include a pointer, a clear protective cover (not shown), and indices or scale markings.

FIG. 8 illustrates a side view, in partial cutaway, of the distal tip of a steerable transseptal needle 800 comprising an outer tube 102, an inner tube 116 further comprising a lumen 412, a hollow punch shaft 802 further comprising a punch lumen 816, a cutting blade 804 further comprising a sharp edge 808, a spring 814, and a hinge pin 810, a slot 812 in the side wall of the hollow punch shaft 802, and a sharp nose cone or tip 806.

The cutting blade 804 is disposed within the lumen 816 of the punch tube 802. The cutting blade comprises at least one sharp edge 808. The cutting blade 804 is hinged about pin 810 and can rotate such that its sharp edge 808 projects laterally outward from the punch shaft tube 802 through slot 812. The punch tip 806 is affixed to the distal end of the punch tube 802 or it can be integrally formed therewith. The punch tip 806, the inner tube 116, the outer tube 102, the hinge pin 810, the cutting blade 804, the spring 814 and the punch shaft 802 can comprise materials such as but not limited to, stainless steel, tantalum, platinum, platinum iridium, titanium, nitinol, PEEK, and the like.

The cutting blade 804 is illustrated with its hinge pin 810 proximally situated such that the cutting blade rotates radially outward biased by the spring 814 against the lumen wall 412. This configuration allows for simple retraction of the cutting blade 804 by proximal withdrawal of the punch shaft 802 within the lumen 412. The sprung cutting blade 804 is shoehorned within the lumen 412 in this configuration. Other configurations are also possible, for example using active control mechanisms to activate and withdraw the blade 804. The cutting blade 804 allows the system to create a larger, or wider, incision in the tissue being penetrated than would be otherwise possible with the punch tip 806, alone.

FIG. 9 illustrates a side view, in partial cutaway, of the distal tip of a steerable transseptal needle 900 comprising an outer tube 102, an inner tube 116 further comprising a lumen 412 and a distal point 408, a punch shaft 902, a grooved punch end 904 further comprising a plurality of circumferential grooves 908, a plurality of groove edges 910, and a sharp punch tip 906. The punch shaft 902 is slidably disposed within the inner tube lumen 412 and controlled by a mechanism at the proximal end of the steerable transseptal needle 900 to oscillate on axis proximally and distally. Such axial movement can be manually controlled or it can be controlled by an actuator, motor, or the like.

The edges 910 can be configured with maximum sharpness and the width of the slots or grooves 908 can be such that tissue can protrude into the slots 908 for maximum cutting effect when the punch grooves are withdrawn into the lumen 412, past the distal end tip 408. The groove widths can range from about 0.010 to about 0.100 inches or greater. The number of grooves 908 can range from at least 1 to a maximum of 10 or more. The depth of the grooves 908 can range from about 0.002 inches to about 0.010 inches with the practical depth determined by the strength of the remaining shaft diameter at the bottom of the grooves 908 and the need to maintain high strength in that area so that no structural failures might occur because of the grooving. In the illustrated embodiment, the shaft 902 diameter is about 0.018 inches in diameter while the outside diameter of the distal grooved area 904 is about 0.022 inches in diameter. However, the shaft 902 and the grooved area 904 can have the same or similar diameters. The shaft diameters as well as groove and thread depths are similar or the same for all the piercing stylet or punch systems described within this document. The shaft diameter is beneficially smaller in diameter than the tip to prevent or minimize binding in the event that the lumen 412 undergoes partial collapse when the inner tube is bent or articulated. The nose cone 906 can comprise shapes such as, but not limited to, trocars, tapered conics, spade bits, threads, and the like. Thus, for example, the 0.018 diameter wire does not bind but 0.021 or 0.022 diameter wire will bind when the distal end is articulated or bent.

The circumferential grooves 908 can, in other embodiments, appear as linear grooves along one or more sides of the punch shaft 902 appearing a bit like gills on a fish. Linear grooves can be applied in various locations to provide for cutting in more than one circumferential region of the punch shaft 902.

In these additional embodiments, a piercing stylet with a sharpened tip is configured to rotate about its longitudinal axis in a continuous, intermittent, or temporary manner. The distal tip of the piercing stylet can comprise a screw thread or it can be formed into a corkscrew by spiraling the tip wire. The corkscrew can comprise a sharpened tip with a conical, trocar, or other configuration. The distal piercing tip of the stylet can comprise a flattened spade bit with a sharpened center and wings configured for gouging out tissue as the spade big spins about its axis. The spade bit can comprise between about 1 and 8 flutes with a preferred flute count of 2 to 4. The rotating, piercing stylet distal tip can comprise a plurality of configurations including, for example, a trocar tip followed by a spade bit. The spade bit can be described as a point with more distally located lateral wings which can be sharpened at their distal aspects, proximal aspects, radial extents, or a combination thereof. The spade bit wings can be configured to fold to a first smaller diameter for insertion through the lumen of the needle and then expand outward to a second, larger diameter, following exposure beyond the distal end of the needle. The piercing stylet is slidably inserted through the main lumen of a steerable transseptal needle. The rotation can be generated by an electric motor, a pneumatic motor, the vibration generator (108), or the like, or it can be turned manually by the operator by way of a knob at the proximal end of the needle. By rotating and drilling into the myocardium or other tissue, the threaded or corkscrew stylet can not only penetrate the fossa tissue but also pull it toward the needle to reduce the amount of tenting required to cross the fossa. The manual operation can comprise advancing longitudinally along with a concurrent rotation of the piercing stylet.

In practice, the rotating piercing stylet can be set in rotary or oscillatory motion using motors, actuators, manual force, spring loaded power, or the like (108). The handle of the piercing stylet is advanced distally to cause the rotating or oscillating tip to be exposed beyond the distal end of the needle. The tip of the piercing stylet is configured to be sharp and penetrate the tissue. The facets or flats that are located proximal to the distal tip are rotated or oscillated to cut or tenderize the tissue to allow for larger diameter structures to be passed through the hole created by the piercing stylet. These larger diameter structures include the transseptal needle, the dilator of the transseptal introducer, and the transseptal introducer itself.

In other embodiments, the hub of the piercing stylet can comprise fully manual or manually assisted function. The push button, for example can be separated from the shaft by a bearing that allows for rotation but retains the push button affixed to the shaft by a bearing or bushing system. The shaft can be routed through a threaded guide that causes it to rotate as the push button is advanced distally. A return spring can allow for retraction of the shaft and commensurate rotation due to the threaded guide or spiral track around the shaft, once the push button is released. This manual system can be beneficially used to actuate a spade bit or circumferentially grooved, or trocar style piercing stylet tip into tissue.

In accordance with current terminology pertaining to medical devices, the proximal direction will be that direction on the device that is furthest from the patient and closest to the user. The distal direction is that direction closest to the patient and furthest from the user. These directions are applied along the longitudinal axis of the device, which is generally an axially elongate structure having one or more lumens or channels extending through the proximal end to the distal end and running substantially the entire length of the device.

The punch may comprise an inner core wire or stylet, an inner tube and an outer tube. In The stylet can be removable or non-removable. The punch further comprises a hub at its proximal end which permits grasping of the punch and also includes a stopcock or valve to serve as a lock for the stylet, or inner core wire, as well as a valve for control of fluid passage into and out from the innermost lumen within which the stylet or inner core wire resides. The proximal end may further comprise one or more control handles to manipulate the amount of articulation at the distal end of the catheter. The proximal end further can be terminated with one or more female Luer or Luer lock ports, which are suitable for attachment of pressure monitoring lines, dye injection lines, vacuum lines, a combination thereof, or the like.

Various vibrations generators can be used. A vibrating motor such as those found on cell phones to generate a vibrate of buzz function, can be used for the purpose. A sonic transducer can be separately affixed to the endoluminal punch or it can be built therein integrally. The power supply for the vibration device can be integral to the hub or separate and operably connected to the vibration device by way of a bus, electrical lead, or the like.

The vibration generator can comprise a moving coil such as found in a loudspeaker, a linear actuator, an ultrasound transducer, which operates at frequencies above those of human hearing, or the like. In an embodiment, high intensity focused ultrasound (HIFU) can be used to power the tip of the punch to enhance tissue cutting or piercing. The vibration generator, in other embodiments, can comprise a rotary electric motor affixed to an off-center cam or weight that spins with the axis of the motor, thus generating internal vibration at the motor shaft. In other embodiments, the vibratory or reversing linear transducer can comprise a rotary motor and a crankshaft or camshaft that comprises a wheel and a shaft rotationally affixed thereto.

To add additional functionality, the endoluminal punch 100 can comprise monitoring systems to measure, display, announce, record, or evaluate operating parameters of the punch including force or energy applied, frequency, location, tissue contact, successful tissue penetration, and the like. The monitoring systems can comprise standard computers, tablet computers, cell phones, and the like. In an embodiment, the vibratory endoluminal punch 100 can comprise strain gauges to measure the force being applied by the user to push on the needle. A human interface can, in other embodiments, comprise audible feedback such as a simple beep or tone, or it can be more sophisticated and provide information using language callouts such as force, turns, energy, torque, or the like.

To provide additional functionality, a pressure monitoring device such as a catheter tip pressure transducer, or a pressure line terminated by a pressure transducer, can be affixed to a quick connect, generally a Luer fitting, at the proximal end of the punch hub. By monitoring pressure, it is possible to determine when the distal end of the punch has passed from, for example, the right atrium into the left atrium, because the pressure versus time curves in these two chambers are measurably, or visually, different. The proximal end of the hub further can comprise provision for attachment to a dye injection line for use in injecting radiographic contrast media through the central lumen of the punch. Typically a manifold can be attached to the Luer fitting on the proximal end of the hub, the manifold allowing for pressure monitoring, for example on a straight through port, and for radiopaque dye injection, for example through a side port. A stopcock, or other valve, can be used to control which port is operably connected to the central lumen of the punch.

The inventions described above may be embodied in other specific forms without departing from its spirit or essential characteristics. The described embodiments are to be considered in all respects only as illustrative and not restrictive. For example, control over energy applied to the apparatus, relative force application to the inner and outer tubes and control rods and rotation of the system about its longitudinal axis can be controlled by electromechanical actuators and logic controllers, or the like. The entire system hub can be affixed into a holding system, which is affixed relative to the location of the patient to allow for stability and fine control over location and various operations in a manner similar to other robotic devices. An electrical power supply can be provided and operably connected to an electrical bus that passes current through, and heats, a resistive element (e.g. a length of nickel chromium wire) at or near the distal tip of the needle, the center punch piercing stylet, or both. In the case of the heatable piercing stylet, the shaft can be beneficially tubular to allow for a lumen to contain an electrical bus. The electrical bus can comprise two conductors or a single conductor since parts of the shaft could serve as one of the conductors. This power supply can be AC or DC. The power supply can be in the form of batteries affixed to and operably connected to the hub. A switch can be used to turn the power on such that heating occurs at the distal tip of the device to denature tissue and provide for increased ease of penetration, with the needle alone or with the needle and the piercing stylet or center punch. Of course the device can also be used with radiofrequency (RF) energy (monopolar or bipolar) such as is provided by a Bovie (e.g. Valleylab, Inc.) by placing a grounding pad against the patient's skin and applying such energy to the needle shaft or other electrically conductive part connected thereto. The shaft does not need to be insulated (although it could be as long as the metal tip is not insulated) because the needle generally resides within the lumen of an introducer which is electrically insulated.

What is claimed is:

1. An endoluminal punch comprising:
a first tube having a proximal end, a distal end, and a lumen extending from said proximal end to said distal end, said first tube having a sharp distal tip;
a stylet having a proximal end and a distal end, said stylet slidably and rotatably disposed within the lumen of the inner tube, with a spiral wire section and a sharp distal tip at the distal end of the stylet, said spiral wire section being resilient and configured to be inserted through, and withdrawn through the first tube.

2. The endoluminal punch of claim 1, further comprising:
a motor operably connected to the proximal end of the stylet, said motor operable to rotate the stylet within the lumen of the first tube.

3. The endoluminal punch of claim 2, wherein:
the motor comprises a hollow shaft, and the proximal end of the stylet is disposed within said hollow shaft and extends proximally from the motor, and the stylet is longitudinally translatable within the hollow shaft.

4. The endoluminal punch of claim 1, further comprising:
a second tube, disposed about the first tube, said second tube having a proximal and a distal end, wherein the first tube is longitudinally fixed to the second tube proximate the distal end of the second tube;
whereby the first tube may be tensioned relative to the second tube to cause deflection of a distal portion of the endoluminal punch.

5. An endoluminal punch comprising:
a first tube having a proximal end, and distal end, and a lumen extending from said proximal end to said distal end;
a stylet disposed within the lumen of the first tube, with a distal tip extending distally beyond the distal end of the first tube; wherein
the first tube comprises a beveled distal tip; and
the stylet comprises an eccentric radial protrusion, proximate the beveled distal tip of the first tube; whereby
the stylet may be rotated to cause vibration of the beveled distal tip.

6. The endoluminal punch of claim 5 further comprising:
a motor coupled to the stylet, operable to rotate the stylet to cause the radial protrusion to repeatedly impinge upon a distal portion of the beveled distal tip, to cause radial vibration of the beveled distal tip.

7. The endoluminal punch of claim 5 further comprising:
a vibration generator longitudinally fixed to the first tube, and coupled to the stylet, said vibration generator operable to cause reciprocating motion of the stylet within the lumen of the first tube.

8. The endoluminal punch of claim 5 wherein:
the stylet has a uniform diameter proximate the distal end of the first tube, and the eccentric radial protrusion comprises a curvature in the stylet, and the stylet is resiliently deformable such that the distal tip of the stylet, including the eccentric radial protrusion, may be drawn proximally through the first tube.

9. The endoluminal punch of claim 5 wherein:
the stylet has a non-uniform diameter proximate the distal end of the first tube.

10. The endoluminal punch of claim 5 wherein:
the stylet has a non-uniform cross-sectional weight distribution proximate the distal end of the first tube.
